(19) United States
(12) Patent Application Publication
(10) Pub. No.: US 2001/0012899 A1
(43) Pub. Date: Aug. 9, 2001

(54) Oligomeric methine dyes

(75) Inventor: Jurgen Geiwiz, Lorrach-Hauingen (DE)

Correspondence Address: CLARIANT CORPORATION, 4331 CHESAPEAKE DR, ATTN: INDUSTRIAL PROPERTY DEPT, CHARLOTTE, NC, 28216, US

(73) Assignee: CLARIANT FINANCE ( BVI) LIMITED

(21) Appl. No.: 09/792,754

(22) Filed: Apr. 18, 2001

(30) Foreign Application Priority Data
Nov. 18, 1999 (CH) 1999 2102/99

(51) Int. Cl. C07D 207/10

(52) U.S. Cl. 548/558

(57) ABSTRACT

Oligomeric methine dye compounds obtainable from a reaction of a diamine with the substances mentioned in the claims, a storage-stable formulation thereof, their use for dyeing organic substrates and substances dyed with such dyes.

### SUMMARY OF THE INVENTION

The present invention comprises compounds or their mixtures obtainable by a diamine being reacted in a first step with at least 2.5 equivalents of a compound containing two leaving groups or groups capable of N-alkylation, in a second reaction step with an aromatic N-containing heterocyclic compound or a nucleophilic compound or a mixture thereof, in a third reaction step with an aldehyde and finally admixed in a fourth reaction step with an inorganic or organic acid.

### DESCRIPTION OF THE PREFERRED EMBODIMENTS

Preferred compounds or mixtures thereof are obtainable by reacting in a first reaction step a diamine of the following formula (I)

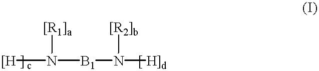

where

R₁ and R₂ are independently substituted or unsubstituted C₁₋₄alkyl or substituted or unsubstituted phenyl,

B₁ is C₂₋₁₀alkylene, which may optionally be interrupted by one or more atoms selected from the group consisting of N, O and S, preferably by O, and which may additionally be substituted,

a, b, c and d are each 0, 1, or 2, subject to the provisos that the sum a+c=2 and the sum b+d=2,

at a temperature of 10-50° C., preferably 15-40° C., particularly preferably 20-30° C., with at least 2.5 equivalents of a compound of the formula XB₂Y where

B₂ is C₁₋₁₀alkylene, which may optionally be interrupted by one or more atoms selected from the group consisting of N, O and S, preferably by O, and which may additionally be substituted, and

X and Y are independently a leaving group, preferably halogen, particularly preferably C1, or a group suitable for alkylating N, for example epoxide, and

in a second reaction step the reaction solution being reacted with a compound D or a plurality of compounds D,

where D is one of the following compounds:

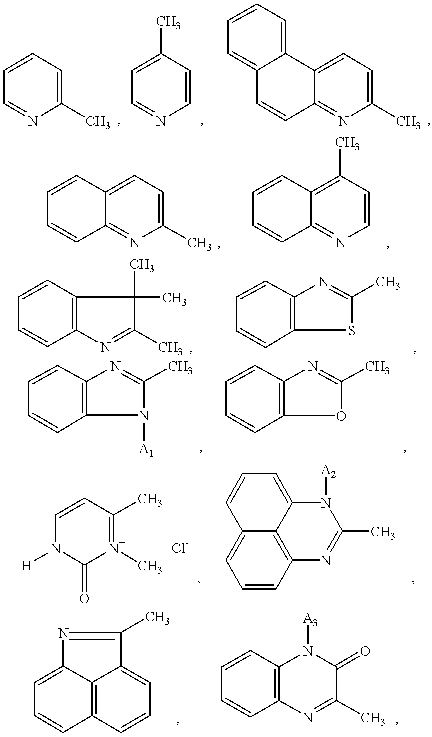

where A₁, A₂ and A₃ are independently C₁₋₄alkyl, benzyl, cyclohexyl, hydroxyalkyl or C₂₋₃alkenyl and the rings of the above radicals may be unsubstituted or substituted by halogen, cyano, C₁₋₄alkyl, C₁₋₄hydroxyalkyl or C₁₋₄alkoxy, or D is a nucleophilic compound, especially HO⁻, N,N-di-C₁₋₄alkylamines or cycloamines selected from the group consisting of morpholine, piperazine and piperidine,

at a temperature of 70-150° C., preferably 80-120° C., particularly preferably 90-110° C., and in a third reaction step the reaction solution being reacted with a compound of the formula (II)

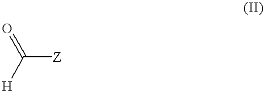

or a mixture thereof,

where Z is phenyl unsubstituted or substituted by hydroxyl, alkoxycarbonyl, N-substituted or unsubstituted carbamoyl, alkyl, alkoxy, amino or substituted amino, unsubstituted or alkyl-, alkoxy-, hydroxyl-, carboxyl- or (substituted amino)-substituted naphthyl, styryl, furyl, thienyl, pyridyl, indolyl, benzofuryl, benzothienyl, pyrazolyl, oxazolyl, thiazolyl, triazolyl, oxadiazolyl, thiadiazolyl, benzimidazolyl, indazolyl, benzoxazolyl, benzothiazolyl, carbazolyl, phenothiazinyl or phenoxazinyl, especially methoxycarbonylphenyl, ethoxycarbonylphenyl, hydroxyphenyl, dihydroxyphenyl, aminocarbonylphenyl, dimethylamino-carbonylphenyl, diethylaminocarbonylphenyl, methoxyphenyl, dimethoxyphenyl, trihydroxyphenyl, trimethoxyphenyl, ethoxyphenyl, butoxyphenyl, phenoxy-phenyl, aminophenyl, methylaminophenyl, ethylaminophenyl, benzylaminophenyl, butyl-aminophenyl, phenylaminophenyl, cyanoethylaminophenyl, dimethylaminophenyl, dimethylaminomethylphenyl, dimethylaminomethoxyphenyl, dimethylaminocarbomethoxyphenyl, diethylamino-phenyl, diethylaminomethylphenyl, ethoxydiethylaminophenyl, dipropylamino-phenyl, di-n-butylaminophenyl, dibenzylaminophenyl, di-□-cyanoethylaminophenyl, di-□-methoxyethylaminophenyl, N-methyl-N-ethyl-aminophenyl, N-butyl-N-methylaminophenyl, N-methyl-N-benzylaminophenyl, N-ethyl-N-methoxybenzyl-aminophenyl, N-cyclohexyl-N-benzylaminophenyl, N-□-cyanoethyl-N-benzylamino-phenyl, N-□-ethoxyethyl-N-benzylaminophenyl, N-□-methoxycarbonylethyl-N-benzylaminophenyl, di-benzylaminochlorophenyl, dibenzyl-aminomethoxyphenyl, dibenzyl-aminomethylphenyl, N-methyl-N-□-cyanoethylamino-phenyl, N-ethyl-N-□-cyanoethylaminophenyl, N-propyl-N-□-cyanoethylaminophenyl, N-butyl-N-□-cyanoethylaminophenyl, N-methyl-N-□-methoxycarbonyl-ethylamino-phenyl, N-methyl-N-□-ethoxycarbonylethylaminophenyl, N-methyl-N-□-carbamoylethylaminophenyl, N-methyl-N-□-dimethylcarbamoylethylaminophenyl, N-ethyl-N-□-methoxycarbonyl-ethylaminophenyl, N-ethyl-N-□-ethoxycarbonylethylamino-phenyl, N-ethyl-N-□-carbamoylethylaminophenyl, piperidinophenyl, pyrrolidino-phenyl, morpholino-phenyl, thiamorpholinophenyl, piperazinophenyl, N-methyl-piperazinophenyl, N-benzylpiperazino-phenyl, N-methyl-N-phenylaminophenyl, N-cyanoethyl-N-phenyl-aminophenyl, diphenyl-aminophenyl, N-methyl-N-4-ethoxy-phenylaminophenyl, N-methyl-N-4-methoxyphenyl-aminophenyl, N-methyl-N-4-methylphenylaminophenyl, N-methyl-N-2-methylphenylamino-phenyl, N-methyl-N-cyanomethylaminophenyl, N-ethyl-N-cyanomethylaminophenyl, N-benzyl-N-cyanomethylaminophenyl, N-□-cyanoethyl-N-cyanomethylaminophenyl, N-methyl-N-□-acetoxyethylaminophenyl, N-ethyl-N-□-acetoxyethylaminophenyl, N-benzyl-N-□-propoxyethylaminophenyl, N-ethyl-N-□-oxethylaminophenyl, N-methyl-N-□-oxethylaminophenyl, dimethylamino-hydroxyphenyl, diethylaminohydroxyphenyl, dibenzyl-aminohydroxyphenyl, dimethylaminoacetylaminophenyl, diethylamino-acetylaminophenyl, N-ethyl-N-□-dimethyl-aminoethylaminophenyl, N-methyl-N-□-dimethylaminoethylamino-phenyl, N-benzyl-N-□-dimethylaminoethylaminophenyl, N-□-cyanoethyl-N-□-dimethylaminoethylaminophenyl, N-□-methoxycarbonylethyl-N-□-dimethylaminoethylaminophenyl, N-□-oxethyl-N-p-dimethyl-aminoethylaminophenyl, N-□-methoxy-ethyl-N-□-dimethyl-aminoethylaminophenyl, N-ethyl-N-□-diethylaminoethylaminophenyl, N-ethyl-N-□-di-enzylaminoethylaminophenyl, N-ethyl-N-□-piperidinoethylaminophenyl, N-benzyl-N-□-morpholinoethylaminophenyl, N-ethyl-N-□-trimethylammonium ethylaminophenyl chloride, N-methyl-N-□-trimethyl-ammonium ethylaminophenyl chloride, N-methyl-N-□-diethylbenzyl-ammonium ethyl-aminophenyl chloride, N-benzyl-N-□-dimethylbenzylammonium ethylaminophenyl chloride, N-ethyl-N-□-pyridinium ethylaminophenyl chloride, dimethylaminonaphthyl, diethylamino-naphthyl, dibenzyl-aminonaphthyl, tolylmethylaminonaphthyl, ethoxyphenyl-methylaminonaphthyl, hydroxynaphthyl, hydroxymethoxycarbonylnaphthyl, methoxycarbonylmethoxynaphthyl,

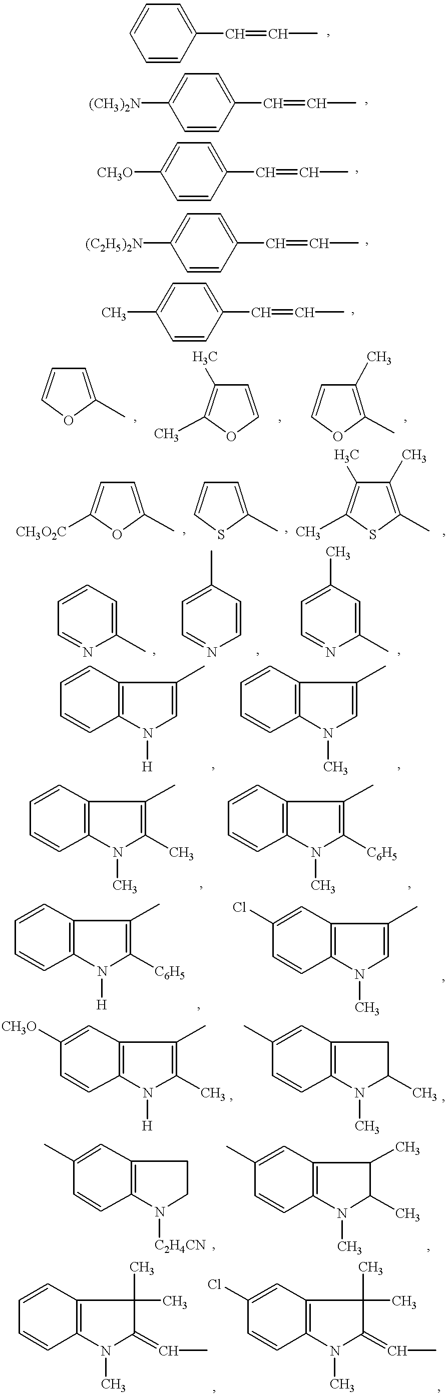

at a temperature of 70-150° C., preferably 80-120° C., particularly preferably 90-110° C., and in a fourth reaction step the reaction solution being admixed with an organic or inorganic acid, especially hydrochloric acid, sulphuric acid, phosphoric acid, formic acid, acetic acid, propionic acid, glycolic acid, citric acid, lactic acid and gluconic acid, preferably formic acid or acetic acid.

More preferred compounds or mixtures are obtainable when

R₁ and R₂ are independently methyl or ethyl,

B₁ is C₂₋₆-alkylene, which may optionally be interrupted by one or more atoms selected from the group consisting of N, O and S, preferably by O, and which may additionally be substituted,

a and b are independently 0 or 1,

B₂ is C₁₋₄ alkylene, which may optionally be interrupted by one or more atoms selected from the group consisting of N, O and S, preferably by O, and which may additionally be substituted,

X is a halogen, preferably C1,

Y is an epoxide,

D is

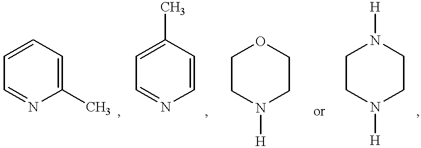

Z is

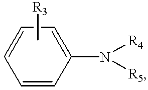

, where the asterisk * symbolizes the bond to the —CHO group and R₃, R₄ and R₅ are independently H or a C₁₋₄-alkyl group, preferably R₃ is H or —CH₃ and R₄═R₅═ —CH₃ or —CH₂CH₃.

Likewise more preferred compounds or mixtures are obtainable when the reaction solution is reacted with a mixture of compounds D in the second reaction step.

Ideally the reactions take place in a polar solvent, especially in alcohols, for example methanol, ethanol or glycols.

The new compounds obtained can directly be used as dyes or in the form of aqueous, for example, concentrated stable solutions for dyeing fibre material of all kinds, cellulose, cotton, keratinous fibres, for example hair, or leather, but in particular paper or paper products, especially woody paper, so-called groundwood, or else bast fibres such as hemp, flax, sisal, jute, coir or straw.

The compounds of the invention are notable for excellent water solubility.

The dyes of the invention can also be mixed with suitable dyes of the same or other dye classes and be used for dyeing and printing the abovementioned materials. More particularly, cationic or basic dyes, for example methine or azo dyes, are suitable for use in mixtures with the dyes of the invention.

The invention also provides for the use of an inventive compound or mixtures thereof for producing a storage-stable, liquid-aqueous dye preparation which includes a compound or mixtures thereof prepared according to the invention and/or their salts.

The invention also provides for the use of the compounds prepared according to the invention or of mixtures thereof or for the use of the storage-stable, liquid-aqueous dye preparation for dyeing or printing organic substrates, especially cellulose, cotton, keratinous substrates, for example hair, or leather, preferably paper or paper products, particularly preferably woody paper, so-called groundwood, or else bast fibres such as hemp, flax, sisal, jute, coir or straw.

The invention also provides organic substrates, especially cellulose, keratinous substrates, for example hair, or leather, preferably paper or paper products, particularly preferably woody paper, so-called groundwood, or else bast fibres such as hemp, flax, sisal, jute, coir or straw which have been dyed or printed with compounds prepared according to the invention or with mixtures thereof or with a storage-stable, liquid-aqueous dye preparation according to the invention.

The examples which follow serve to illustrate the invention. In the examples, parts are weight per cent, unless otherwise stated; the temperatures are reported in degrees Celsius.

### EXAMPLES

### Example 1

139 parts of (±)-2-chloromethyloxirane (XB₂Y) are gradually added dropwise with gentle cooling to a solution of 58 parts of 1,6-diaminohexane (I) in 300 parts of 1,2-propylene glycol. On completion of the addition the mixture is subsequently stirred at 20-30° C. for 3 hours, at which point 93 parts of 4-picoline (D) are added. The solution is gradually heated to 90-100° C. and stirred at that temperature for 3 hours. Then 25 parts of piperazine (D) are added, and stirring is continued at 90-100° C. for a further 30 minutes. 116 parts of 4-dimethylaminobenzaldehyde (II) are then added, and stirring is continued at about 100° C. for a further 2 hours. Finally the batch is diluted with a mixture of 450 parts of water and 310 parts of acetic acid.

The about 28% strength dye solution dyes woody materials in brilliant orange shades with minimal wastewater contamination.

Example 1 was repeated to prepare further dyes whose hues in paper dyeing are reported in the table which follows:

### Table 1

### Examples 2-26

| Ex. | (I) | XB₂Y | D | D | (II) | Hue |
|---|---|---|---|---|---|---|
| 2 | 1,6-diaminohexane | (±)-2-chloromethyl- | 4-picoline | piperazine | 4-diethylaminobenzaldehyde | brilliant red |
| | | oxirane | | | | |
| 3 | Ditto | ditto | 2-picoline | ditto | ditto | brilliant red |
| 4 | Ditto | ditto | 4-picoline | morpholine | ditto | brilliant red |
| 5 | Ditto | ditto | ditto | piperazine | 4-diethylamino-2-methyl- | brilliant bluish red |
| | | | | | benzaldehyde | |
| 6 | Ditto | ditto | ditto | pyrrolidine | 4-dimethylaminobenzaldehyde | brilliant yellowish red |
| 7 | 1,3-diaminopropane | ditto | ditto | piperazine | 4-diethylaminobenzaldehyde | brilliant red |
| 8 | Ditto | ditto | ditto | ditto | 4-dimethylaminobenzaldehyde | brilliant orange |
| 9 | Ditto | ditto | 2-picoline | ditto | ditto | brilliant orange |
| 10 | Ditto | ditto | 4-picoline | ditto | N-methyl-N-□-cyanoethyl-4- | brilliant red |
| | | | | | aminobenzaldehyde | |
| 11 | Ditto | ditto | ditto | ditto | N-ethyl-N-(2′-hydroxyethyl)-4- | brilliant red |
| | | | | | amino-2-methyl-benzaldehyde | |
| 12 | 1,2-diaminoethane | ditto | ditto | ditto | 4-dimethylaminobenzaldehyde | brilliant orange |
| 13 | Ditto | ditto | ditto | ditto | 4-diethylaminobenzaldehyde | brilliant red |
| 14 | 1,4-diaminobutane | ditto | ditto | ditto | ditto | brilliant red |
| 15 | Ditto | ditto | ditto | ditto | 4-dimethylaminobenzaldehyde | brilliant orange |
| 16 | 1,5-diaminopentane | ditto | ditto | ditto | ditto | brilliant orange |
| 17 | 1,2-diaminoethane | (±)-2-chloromethyl- | 2-methyl-quinoline | piperazine | 4-diethylaminobenzaldehyde | brilliant violet |
| | | oxirane | | | | |
| 18 | ditto | ditto | ditto | ditto | 4-dimethylaminobenzaldehyde | brilliant violet |
| 19 | ditto | ditto | 4-methyl-quinoline | ditto | ditto | brilliant violet |
| 20 | ditto | ditto | ditto | ditto | 4-diethylaminobenzaldehyde | brilliant violet |
| 21 | 1,4-bis-(3-amino- | ditto | ditto | ditto | ditto | brilliant red |
| | propyloxy)-butane | | | | | |
| 22 | 1,3-diaminopropane | ditto | 2,3,3-trimethyl-3H- | ditto | ditto | violet |
| | | | indole | | | |
| 23 | ditto | ditto | ditto | ditto | 4-dimethylaminobenzaldehyde | violet |
| 24 | 1,6 diaminohexane | ditto | 4-picoline | ditto | N-methyl-N-□-cyanoethyl-4- | brilliant orange |
| | | | | | aminobenzaldehyde | |
| 25 | ditto | ditto | ditto | ditto | 3,3′-(4-formylphenylimino)- | yellow |
| | | | | | dipropionitrile | |
| 26 | ditto | ditto | ditto | ditto | 1,3,3-trimethyl-2-methylen-□- | violet |
| | | | | | aldehyde | |

The use examples which follow serve to illustrate the invention. In the examples, parts are weight per cent, unless otherwise stated; the temperatures are reported in degrees Celsius.

### Use Example A

A hollander is used to grind 70 parts of chemically bleached sulphite softwood cellulose and 30 parts of chemically bleached sulphite birchwood cellulose into 2000 parts of water. 0.2 part of the dye from Example 1 is sprinkled in. After a mixing time of 20 minutes paper is made from the stuff. The thusly obtained absorbent paper has a yellowish red colour. The wastewater is colourless.

### Use Example B

0.5 part of the dye solution of Example 1 is poured into 100 parts of bleached sulphite cellulose ground with 2000 parts of water in a hollander. Mixing for 15 minutes was followed by sizing. Paper made from this material has a yellowish red hue.

### Use Example C

An absorbent web of unsized paper is pulled at 40-50° C. through a dye solution of the following composition:

0.5 part of the dye of Example 1,

0.5 part of starch and

99.0 parts of water

Excess dye solution is squeezed off by two rolls. The dry paper web has a yellowish red colour.

The method of Use Examples A to C is also suitable for dyeing with the dyes of the other examples.

### Use Example D

15 kg of wastepaper (woody), 25 kg of bleached groundwood and 10 kg of unbleached sulphate pulp were beaten in a pulper to form a 3% aqueous pulp suspension. The pulp suspension was diluted to 2% in a dyeing vat. This suspension was then admixed in succession with 5% of kaolin and 1.25 kg of a 5% acetic acid solution of the dye of Example 1, reckoned on dry total fibre, by stirring. After 20 minutes the pulp in the mixing vat is admixed with 1% (based on absolutely dry fibre) of a resin size dispersion. The homogeneous pulp suspension was adjusted with alum to pH 5 on the paper machine just upstream of the headbox.

The paper machine was used to produce 80 g/m² of yellowish red bag paper with a machine finish.

### Use Example E

A dry stock consisting of 60% groundwood and 40% unbleached sulphite pulp is beaten with sufficient water and ground to 40 SR freeness in a hollander for the dry content to be just above 2.5% and then adjusted with water to a dry content of exactly 2.5% for the high-density pulp.

200 parts of this high-density pulp are admixed with 5 parts of a 0.25% aqueous solution of the dye of Example 1, stirred for about 5 min., admixed with 2% of resin size and 4% of alum, based on dry stock, and again stirred for some minutes until homogeneous. The material is diluted with 500 parts of water to 700 parts by volume and used in a known manner to prepare sheets of paper by drainage on a sheet-former. These sheets of paper have a deep yellowish red colour.

The method of Use Examples D and E is also suitable for dyeing with the dyes of the other examples.

1. Compounds or their mixtures obtainable by a diamine being reacted
in a first step with at least 2.5 equivalents of a compound containing two leaving groups or groups capable of N-alkylation,
in a second reaction step with an aromatic N-containing heterocyclic compound or a nucleophilic compound or a mixture thereof,
in a third reaction step with an aldehyde and finally admixed in a fourth reaction step with an inorganic or organic acid.

2. Compounds or their mixtures according to claim 1 obtainable by reacting in a first reaction step a diamine of the following formula (I) 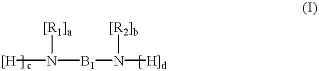
where
R₁ and R₂ are independently substituted or unsubstituted C₁₋₄alkyl or substituted or unsubstituted phenyl,
B₁ is C₂₋₁₀alkylene, which may optionally be interrupted by one or more atoms selected from the group consisting of N, O and S and which may additionally be substituted,
a, b, c and d are each 0, 1, or 2, subject to the provisos that the sum a+c=2 and the sum b+d=2,
at a temperature of 10-50° C. with at least 2.5 equivalents of a compound of the formula XB₂Y where
B₂ is C₁₋₁₀alkylene, which may optionally be interrupted by one or more atoms selected from the group consisting of N, O and S and which may additionally be substituted, and
X and Y are independently a leaving group or a group suitable for alkylating N, and in a second reaction step the reaction solution being reacted with a compound D or a plurality of compounds D,
where D is one of the following compounds: 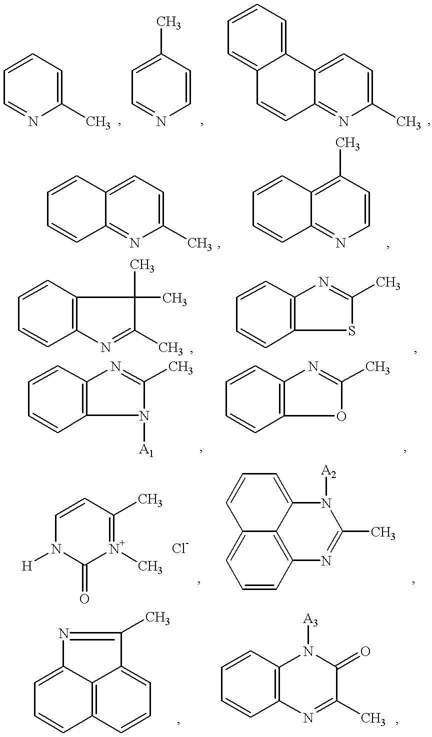
where A₁, A₂ and A₃ are independently C₁₋₄alkyl, benzyl, cyclohexyl, hydroxyalkyl or C₂₋₃alkenyl and the rings of the above radicals may be unsubstituted or substituted by halogen, cyano, C₁₋₄alkyl, C₁₋₄hydroxyalkyl or C₁₋₄alkoxy,
or D is a nucleophilic compound, at a temperature of 70-150° C.,
and in a third reaction step the reaction solution being reacted with a compound of the formula (II) 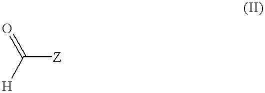
or a mixture thereof,
where Z is phenyl unsubstituted or substituted by hydroxyl, alkoxycarbonyl, N-substituted or unsubstituted carbamoyl, alkyl, alkoxy, amino or substituted amino, unsubstituted or alkyl-, alkoxy-, hydroxyl-, carboxyl- or (substituted amino)-substituted naphthyl, styryl, furyl, thienyl, pyridyl, indolyl, benzofuryl, benzothienyl, pyrazolyl, oxazolyl, thiazolyl, triazolyl, oxadiazolyl, thiadiazolyl, benzimidazolyl, indazolyl, benzoxazolyl, benzothiazolyl, carbazolyl, phenothiazinyl or phenoxazinyl, at a temperature of 70-150° C.,
and in a fourth reaction step the reaction solution being admixed with an organic or inorganic acid.

3. Compounds or mixtures obtainable according to claim 1, characterized in that
R₁ and R₂ are independently methyl or ethyl, B₁ is C₂₋₆ alkylene, which may optionally be interrupted by one or more atoms selected from the group consisting of N, O and S and which may additionally be substituted,
a and b are independently 0 or 1,
B₂ is C₁₋₄ alkylene, which may optionally be interrupted by one or more atoms selected from the group consisting of N, O and S and which may additionally be substituted,
X is a halogen,
Y is an epoxide,
D is 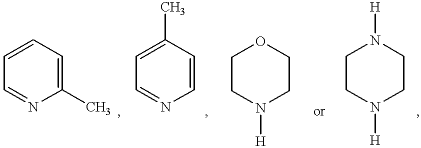
Z is 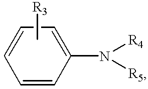
, where the asterisk * symbolizes the bond to the —CHO group and R₃, R₄ and R₅ are independently H or a C₁₋₄-alkyl group and hydrochloric acid, sulphuric acid, phosphoric acid, formic acid, acetic acid, propionic acid, glycolic acid, citric acid, lactic acid or gluconic acid is used in a fourth reaction step.

4. Compounds or their mixtures obtainable according to claim 1, characterized in that the reaction solution is reacted with a mixture of compounds D.

5. Compounds or their mixtures obtainable according to claim 1, characterized in that the reaction temperature is 15-40° C. in the first reaction step and 80-120° C. in the second and third reaction steps.

6. Use of a compound or mixtures thereof according to claim 1 in water-soluble form for producing a storage-stable, liquid-aqueous dye preparation.

7. Use of a compound or mixtures thereof obtainable according to claim 1 of a storage-stable, liquid-aqueous dye preparation for dyeing or printing organic substrates.

8. Use according to claim 7, characterized in that cellulose, cotton, keratinous substrates or leather are used as organic substrates.

9. Organic substrates dyed or printed with compounds or mixtures obtainable according to claim 1 or a storage-stable, liquid-aqueous dye preparation.

10. Organic substrates according to claim 9, characterized in that these are cellulose, cotton, keratinous substrates or leather.
